(19) United States
(12) Patent Application Publication
(10) Pub. No.: US 2006/0198906 A1
(43) Pub. Date: Sep. 7, 2006

(54) Compositions and Method to Increase Serum Levels of HDL cholesterol and Lower the Risk of Cardiovascular Disease

(75) Inventors: Muhammed Majeed, Piscataway, NJ (US); Subbalakshmi Prakash, Piscataway, NJ (US)

Correspondence Address: SABINSA CORPORATION, 70 ETHEL ROAD WEST, UNIT 6, PISCATAWAY, NJ, 08854, US

(73) Assignee: SAMI LABS LIMITED, Bangalore (IN)

(21) Appl. No.: 11/308,111

(22) Filed: Mar. 7, 2006

Related U.S. Application Data
(60) Provisional application No. 60/594,042, filed on Mar. 7, 2005.

(51) Int. Cl. A61K 36/906; A61K 36/67; A61K 36/889; A61K 36/324; A61K 31/198; A61K 31/195

(52) U.S. Cl. 424/725; 424/734; 424/727; 424/756; 514/561; 424/777

(57) ABSTRACT

The invention provides a method for increasing serum high density lipoprotein (HDL) cholesterol levels, and lowering serum levels of Lipoprotein (a) and C-reactive protein, as well as a suitable composition comprising a) a vegetable protein, b) a natural antioxidant compound, c) a natural anti-inflammatory compound, d) a bioavailable source of selenium, e) a natural blend of long chain aliphatic alcohols termed policosanol; and f) a natural bioavailability enhancer.

### CROSS REFERENCE TO RELATED APPLICATIONS

This application claims the benefit of U.S. Provisional patent application No. 60/594,042 filed on Mar. 7, 2005.

### BACKGROUND INFORMATION

Compositions containing natural compounds and nutrients that efficaciously and safely enhance high density lipoprotein cholesterol (HDL-C) levels, are described. These compositions also favorably influence the markers of inflammation and support cardiovascular health and wellness in humans and animals.

Cardiovascular diseases (CVD) continue to remain the leading cause of morbidity and mortality in the developed world, with coronary heart disease (CHD) being rated as the number one killer, and stroke following as the leading third, in the United States. Over the years, research has established the link between dietary fats (lipids), lipid transport and metabolism in the body, atherosclerosis (the progressive narrowing of the arteries over time), and cardiovascular disease. Lipids present in the blood and tissues of the body include cholesterol, cholesterol esters, triglycerides, and phospholipids.

High levels of low-density lipoprotein cholesterol (LDL-C) are particularly correlated with atherosclerosis and cardiovascular disease, based on evidence from clinical studies and epidemiological analysis. Levels of inflammatory markers such as C-reactive protein, homocysteine and low-density lipoprotein (LDL) cholesterol levels are elevated in persons at risk for cardiovascular events.

Currently, the statins are the first choice drugs when treating dyslipidemias, especially in patients with hypercholesterolemia alone or accompanied by hypertriglyceridemia.

Most therapeutic approaches target reducing LDL. However, studies in animal models show that over-expression of apolipoprotein (apo) A-1, the major HDL lipoprotein, inhibits progression and induces regression of atherosclerosis. Therefore HDL is an important target for therapeutic intervention, and low HDL-C levels are a modifiable risk factor for cardiovascular disease.

Current medical opinion considers inflammation of atherosclerotic plaques, and the subsequent formation of blood clots on the surface of these plaques, as critical events that lead to most atherosclerosis induced cardiovascular events.

As National Cholesterol Education Program (from the National Institutes of Health) guidelines have been changed to include a global measure for CHD risk, risk status and treatment measures adopted for some patients, may be different as compared to the earlier guidelines. The number of patients with cholesterol levels that can be classified as abnormal, has now tripled. These guidelines recommend complete lipoprotein profile (total, LDL, HDL, triglycerides) as preferred screening for assessing CHD risk status. LDL remains a primary target of cholesterol-lowering therapy, along with increased emphasis on optimal HDL levels.

Statins are the most powerful drugs for lowering LDL, facilitating dose-related reductions in LDL ranging from 20-60%. Fluvastatin is reported to be the least potent, decreasing LDL levels by only 22-36% at the maximum recommended dosage. Rosuvastatin is reported to be the most potent, reducing LDL levels by up to 65% (in a dose range of 20-80 mg/day), in clinical studies. Statins also have moderate effects on HDL, raising levels by approximately 5%, and decrease triglyceride concentrations to a maximum of about 30%.

Unfortunately, the mechanism of action of statins through inhibition of the mevalonate pathway inhibits the biosynthesis of vital biochemical products of this loop As a result, myalgia and myopathy occur in 2% and 0.5% of patients, respectively, with less than 0.1% of cases progressing into rhabdomyolysis, which may be associated with acute renal failure.

Besides statins, other current lipid-altering agents that lower LDL-C primarily through increased hepatic LDL receptor activity include, bile acid sequestrants/resins and cholesterol absorption inhibitors such as ezetimibe. These are also associated with side effects.

In recent years there has been significant research on the use of safer efficacious natural alternatives and nutritional materials to address lowering LDL-C, raising HDL-C and to help individuals with various types of dyslipidemias. A few examples from published literature are cited here.

Plant sterols/stanols (Metabolism. 52(11):1478-83) polyphenols, natural antioxidant herbal extracts such as curcuminoids from turmeric (Indian J Physiol Pharmacol.; 36(4):273-5), viscous fiber such as oat bran (Ann Nutr Metab. 47(6):306-11), saponin-rich seed extracts from fenugreek (J Assoc Physicians India; 49:1057-61), and seed proteins such as soy protein (Arteriosclerosis, Thrombosis, and Vascular Biology. 2003; 23:2241) have also been shown to be beneficial in lipid lowering.

Guggulsterones the biologically active components of the resin of guggul (Commiphora mukul) used in traditional Ayurvedic medicine to treat inflammation, arthritis, cardiovascular conditions and obesity were recently shown to be antagonist ligands for the bile acid receptor FXR, which is an important regulator of cholesterol homeostasis in the body. (Science; 296:1703-6).

Policosanol (Am HeartJ; 143(2): 356-65) is a natural mixture of higher aliphatic alcohols, found in plant waxes. Sugarcane wax is the common source. The components of policosanol include 1-octacosanol, 1-dotriacontanol, 1-tetracosanol, 1-tetratriacontanol, 1-hexacosanol, 1-heptacosanol and 1-nonacosanol. This mixture of alcohols is clinically proven to be effective in maintaining normal cholesterol levels.

Coconut protein is shown to increase HDL (Indian J Exp Biol. 2001 October; 39(10):1028-34).

L-arginine present in coconut protein is reported to favorably influence blood lipid levels (Indian J. Exptal Biology Vol. 42, January 2004, pp. 53-57). L-arginine favorably influences serum homocysteine levels as well. Homocysteine is a marker of cardiovascular abnormalities. (Journal of Nutrition, 135, pp 212-217).

Curcuminoids extracted from Zingiberaceae species are known natural antioxidants and favorably influence cholesterol levels (Indian J Physiol Pharmacol. 1992 Oct; 36(4):273-5).

Boswellic acids from Boswellia species are reported to lower cholesterol levels in animal models. (Indian J. Pharmacol.—18: 182-183, 1986)

Selenium-enriched garlic is reported to increase HDL-C levels, (Wei Sheng Yan Jiu. 2002 April; 31(2):93-6). Dietary intake of non-fish Se had a positive correlation with HDL cholesterol (Trace Elem. Med Biol. 2002; 16(2):83-90)

Thus natural compounds and nutritional materials that have beneficial effects on blood lipid levels, enhance HDL-C levels and support cardiovascular health are available for use as alternatives or to complement drug therapy.

### SUMMARY OF THE INVENTION

Compositions containing natural compounds and nutrients that efficaciously and safely enhance HDL cholesterol levels, are described. These compositions also favorably influence the markers of inflammation and support cardiovascular health and wellness in humans and animals.

### BRIEF DESCRIPTION OF THE DRAWINGS

FIG. 1 shows the effect of Formulations 1 and 2 on Total Cholesterol in an animal model.

FIG. 2 shows the effect of Formulations 1 and 2 on triglycerides level in an animal model.

FIG. 3 shows the effect of Formulations 1 and 2 on LDL level in an animal model.

FIG. 4 shows the effect of Formulations 1 and 2 on HDL level in an animal model.

FIG. 5 shows the effect of Formulations 1 and 2 on VLDL level in an animal model.

### DETAILED DESCRIPTION

Synergistic Compositions prepared with natural compounds and nutritional ingredients are described in the present invention. These compositions favorably influence HDL-C, Lipoprotein (a), and LDL-C levels, have beneficial effects on inflammation, and provide antioxidant action.

### EXAMPLE 1

Nutritional supplement composition to enhance HDL-C levels The ingredients as listed below were formulated in pharmaceutically acceptable vehicle.

Formulation 1

| | | |
|---|---|---|
| Commiphora mukul Extract(standardized to | 34 | g (68.5% w/w) |
| contain 2.5% guggulsterones) | | |
| Policosanol | 600 | mg (1.2% w/w) |
| Garcinol (40% extract from Garcinia species) | 15 | g (30.2% w/w) |

Animal dose administered: 250 mg/kg body weight

Formulation 2

| | | |
|---|---|---|
| Coconut protein: | 33.3 | g (76.8% w/w) |
| Curcuminoids (from Turmeric | 5.0 | g (11.53% w/w) |
| (Curcuma longa ) roots) | | |
| Acetyl-11-keto-beta-boswellic acid | 5.0 | g (11.53% w/w) |
| Gamma-glutamyl-methyl-L- | 50 | mcg Selenium equivalent |
| selenocysteine | | |

Animal dose administered: 90 mg/kg body weight

### EXAMPLE 2

### Efficacy and Safety Study in Animal Model

The compositions listed in paragraph 21 was tested in an animal model study Study design: Healthy female mice were divided into three groups of 40 mice each (Group A: Control group, Group B: Administered test formulation 1, Group C: Administered Test Formulation 2). Each group was divided into four sub-groups of ten mice each. The segregation was random.

Group B received formulation 1 suspended in 0.5% Carboxymethyl cellulose (CMC) 250 mg/kg body weight along with normal diet.

Group C received Test formulation 2 suspended in 0.5% CMC-90 mg/kg body weight along with normal diet.

The groups were further divided into three sub-groups each. One sub-group from each group was randomly selected to determine blood lipid profile (Total cholesterol, HDL cholesterol and triglycerides). The remaining sub-groups from each group were analyzed at 4 week intervals for blood lipid profiles (4 weeks, 8 weeks, 12 weeks).

Results:

Throughout the experimental period of twelve weeks all the animals tolerated the treatment well. They did not show any change in behavior in feeding, drinking or grooming. They remained alert and were responsive to external stimuli. All the animals survived during the course of treatment.

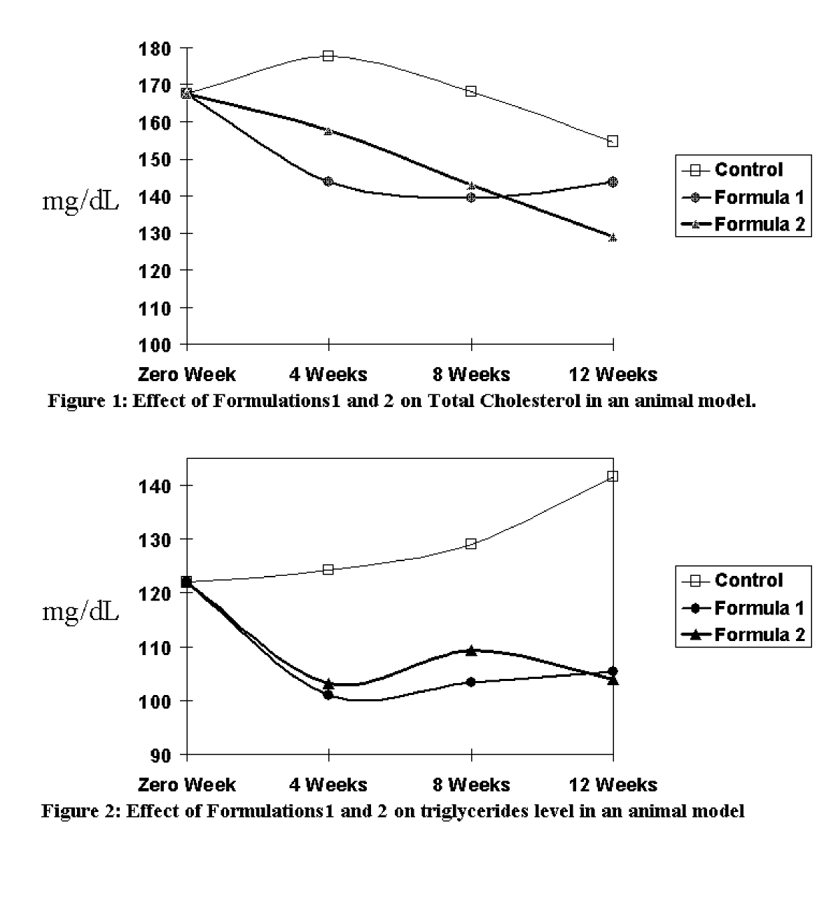

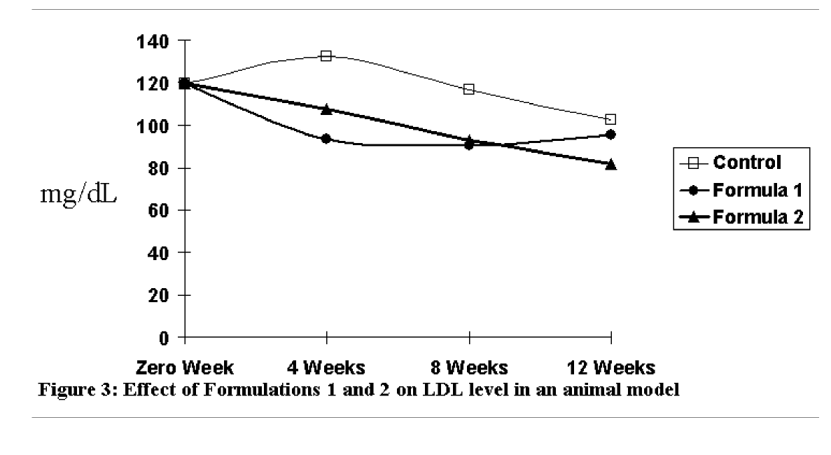

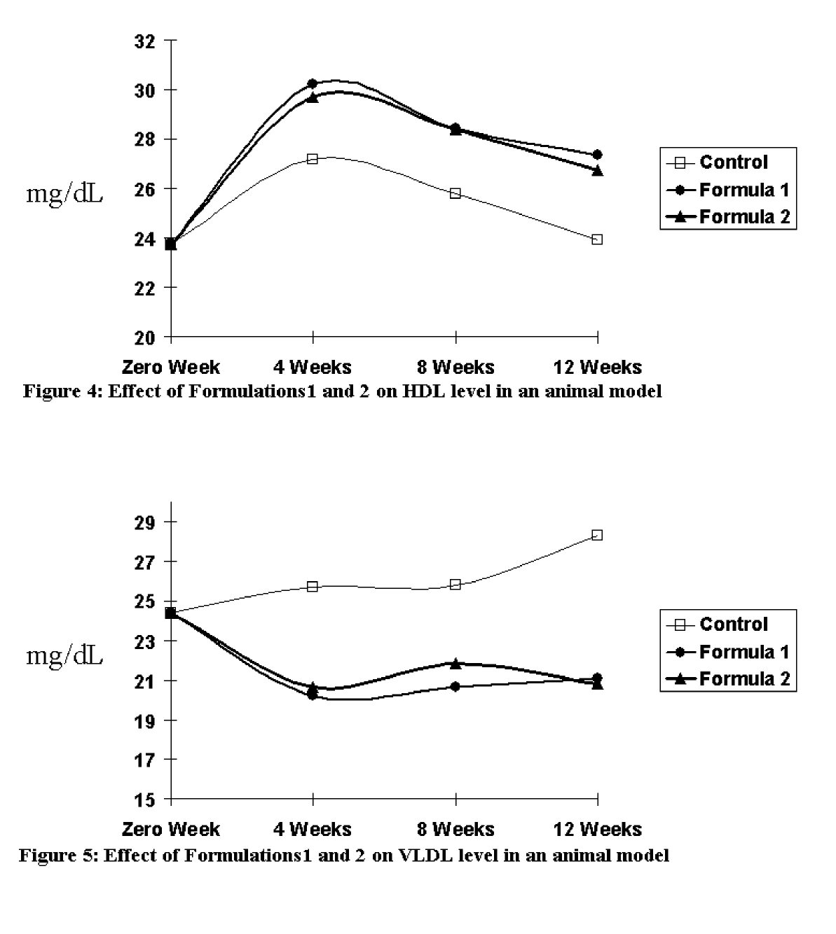

Statistical analysis of the data presented in FIGS. 1-4 revealed the following:

a) Both Formulation 1 and Formulation 2 are effective in favorably influencing blood lipid levels in experimental animals.

b) Periodical analysis through 4, 8 and 12 weeks using inter-group controls indicates that during the first 4 weeks of treatment, formulation 1 has better efficacy in lowering triglycerides, LDL-C, total cholesterol and in increasing HDL-C.

c) At the end of the eighth week, the trend continued as above.

d) At the end of 12 weeks however, formulation 2 was found to more efficaciously reduce total cholesterol and LDL-C as compared to formulation 1. The values of the other parameters were more or less identical for both formulations.

Based on these results, the long term efficacy in lowering total cholesterol and LDL-C of formulation 2 is superior to that of formulation 1. Also formulation 2 efficaciously lowers total cholesterol and LDL-C and raises HDL-C at a lower dose as compared to formulation 1. The dose of formulation 2 needed is 2.8 times lower than that of formulation 1.

### EXAMPLE 3

Evaluation of The safety and efficacy of synergistic compositions of natural compounds and nutritional ingredients in supporting healthy blood lipid levels and cardiovascular health and wellness in human subjects:

A formulation was prepared by dispersing potentially effective amounts of coconut protein, plant based antioxidant and anti-inflammatory compounds, a bioavailable form of nutritional selenium, policosanol, and a natural bioavailability enhancer, in a pharmaceutically acceptable carrier. A suitable oral dosage form was prepared.

| Active Ingredient | Amount/dose | |
|---|---|---|
| Coconut protein | 400 | mg |
| Curcuminoids (from Curcuma longa roots) | 50 | mg |
| Acetyl-11-keto-beta-boswellic acid | 50 | mg |
| Gamma-glutamylmethylselenocysteine | 200 | mcg |
| Policosanol | 5 | mg |
| Black pepper Extract (Bioperine (RTM of | 5 | mg |
| Sabinsa Corporation New Jersey, USA) | | |
| standardized to contain 95% to 98% piperine) | | |

9 individuals (8 males and 1 female) 25 to 55 years of age with total cholesterol levels at baseline ranging from 168 mg/dL to 251 mg/dL participated in the study. The study examined total cholesterol, HDL cholesterol, triglycerides, LDL cholesterol, VLDL cholesterol. Lipoprotein (a), malondialdehyde (a marker of oxidative stress), and C-reactive protein levels (high sensitivity C-reactive protein) in these subjects before treatment, and after 34, 68 days of treatment with 2 tablets/day of the composition

Blood samples were taken at Day 0, Day 34, and Day 68 and evaluated for blood lipid profile and inflammatory markers.

Total cholesterol levels decreased from baseline levels after treatment in all volunteers. The percentage reduction ranged from 10% to 40%. HDL-C levels increased in 7 out of 9 volunteers. The percentage increase from baseline was 1% to 10%. The levels of triglycerides decreased after treatment by 7% to 45% from baseline levels, LDL-C levels decreased from 10% to 45% and VLDL levels decreased by 7% to 45%.

Mean levels of the inflammatory marker C-reactive protein (hsCRP) and malondialdehyde were reduced from baseline levels after 68 days of treatment. The reduction in hsCRP was from an initial value of 1.0±0.5 mg/dL to 0.7±0.3 mg/dL. Malondialdehyde levels decreased from an initial value of 1.2±0.5 mg/dL to 0.7±0.3 mg/dL. Lipoprotein (a). (Lp (a)) levels were reduced by a mean of 5% from the pre-treatment levels.

What is claimed is:

1. A composition comprising: a) a vegetable protein; b) a natural antioxidant compound; c) a natural anti-inflammatory compound; d) a bioavailable source of selenium; e) a natural blend of long chain aliphatic alcohols termed policosanol; and f) a natural bioavailability enhancer.

2. A composition according to claim 1 wherein the vegetable protein is derived from the kernal of Cocos nucifera fruit.

3. A composition according to claim 1 wherein the natural antioxidant compound is a curcuminoid derived from Zingiberaceae species.

4. A composition according to claim 1 whererein the natural anti-inflammatory compound is a boswellic acid derived from Boswellia species.

5. A composition according to claim 1 wherein the bioavailable source of selenium is selected from the group consisting of selenomethionine, methylselenocysteine gamma-glutamyl-methylselenocysteine, a derivative of selenomethionine, a derivative of methylselenocysteine or a derivative of gamma-glutamyl-methylselenocysteine.

6. A composition according to claim 1 wherein the policosanol is extracted from sugarcane wax, rice bran or beeswax.

7. A composition according to claim 1 wherein the natural bioavailabilty enhancer is an extract from Piper nigrum or Piper longum fruit.

8. A tablet, capsule, emulsion, spansule, suspension, syrup, microbead, or granule comprising a composition according to claim 1 in a pharmaceutically acceptable carrier.

9. A nutritional supplement comprising a composition according to claim 1.

10. A food or beverage product comprising a composition according to claim 1.

11. A composition according to claim 1 further comprising a component selected from Commiphora mukul extract, Garcinia cambogia extract, or Garcinia indica extract.

12. A method of reducing serum total cholesterol levels or inhibiting elevated serum total cholesterol, elevated serum triglycerides, elevated serum low density lipoprotein (LDL) cholesterol, and elevated serum very low density lipoprotein (VLDL) cholesterol levels comprising administering to the individual in need thereof an effective amount of a composition comprising: a) a vegetable protein; b) a natural antioxidant compound; c) a natural anti-inflammatory compound; d) a bioavailable source of selenium; e) a natural mixture of higher aliphatic alcohols termed policosanol; and f) a natural bioavailability enhancer.

13. A method of increasing serum levels of high density lipoprotein (HDL) cholesterol comprising administering to the individual in need thereof an effective amount of a composition comprising: a) a vegetable protein; b) a natural antioxidant compound; c) a natural anti-inflammatory compound; d) a bioavailable source of selenium; e) a natural blend of long chain aliphatic alcohols termed policosanol; and f) a natural bioavailability enhancer.

14. A method of reducing serum levels of Lipoprotein (a) or inhibiting elevated serum levels of Lipoprotein (a) comprising administering to the individual in need thereof an effective amount of a composition comprising: a) a vegetable protein; b) a natural antioxidant compound; c) a natural anti-inflammatory compound; d) a bioavailable source of selenium; e) a natural blend of long chain aliphatic alcohols termed policosanol; and f) a natural bioavailability enhancer.

15. A method of reducing serum levels of C-reactive protein or inhibiting elevated serum levels of C-reactive protein comprising administering to the individual in need thereof an effective amount of a composition comprising: a) a vegetable protein; b) a natural antioxidant compound; c) a natural anti-inflammatory compound; d) a bioavailable source of selenium; e) a natural blend of long chain aliphatic alcohols termed policosanol; and f) a natural bioavailability enhancer.
